(19) United States
(12) Patent Application Publication
(10) Pub. No.: US 2024/0363456 A1
(43) Pub. Date: Oct. 31, 2024

(54) GAS-PERMEABLE PACKAGE LID OF CHIP PACKAGE STRUCTURE AND MANUFACTURING METHOD THEREOF

(71) Applicant: Industrial Technology Research Institute, Hsinchu (TW)

(72) Inventors: Lung-Tai Chen, Kaohsiung City (TW); Chin-Sheng Chang, Tainan City (TW); Bor-Shiun Lee, New Taipei City (TW); Liang-Ju Chien, Tainan City (TW)

(73) Assignee: Industrial Technology Research Institute, Hsinchu (TW)

(21) Appl. No.: 18/334,396

(22) Filed: Jun. 14, 2023

(30) Foreign Application Priority Data
Apr. 25, 2023 (TW) 112115267

(51) Int. Cl. H01L 23/04; G01N 33/00; H01L 21/56; H01L 23/06; H01L 23/31

(52) U.S. Cl. CPC H01L 23/04; G01N 33/0027; H01L 21/565; H01L 23/06; H01L 23/315

(57) ABSTRACT

A gas-permeable package lid of a chip package structure and a manufacturing method thereof are provided. The gas-permeable package lid of the chip package structure includes a lid body, an air hole, and a hydrophobic gas-permeable membrane. The lid body is integrally formed with an encapsulation material and has a body portion and a plurality of anchors. The air hole penetrates the body portion of the lid body. The hydrophobic gas-permeable membrane is bonded to the lid body and has a shielding part shielding the air hole and an embedded part embedded in the lid body. The embedded part has an upper surface and a lower surface. The upper surface and the lower surface respectively have a plurality of recesses. The anchors are respectively located in the recesses.

### CROSS-REFERENCE TO RELATED APPLICATION

This application claims the priority benefit of Taiwan application serial no. 112115267, filed on Apr. 25, 2023. The entirety of the above-mentioned patent application is hereby incorporated by reference herein and made a part of this specification.

### TECHNICAL FIELD

The disclosure relates a gas-permeable package lid of a chip package structure and a manufacturing method thereof.

### BACKGROUND

With the emphasis on environmental health and environmental protection, air quality monitoring has been widely implemented, which mainly uses gas sensors for long term environmental sensing to achieve the purpose of monitoring. In order to adapt to long-term operation and different environments or climates, the requirements for the environmental tolerance of gas sensors are gradually increasing.

Most of the hydrophobic gas-permeable membranes of the gas sensors currently available on the market are fixed on the air holes of the gas sensors via adhesion after the chip packaging is completed. However, the hydrophobic gas-permeable membrane must be hydrophobic. This hydrophobic property leads to poor fixation of the adhesive. Therefore, the commercially available gas sensor may have poor bonding of the hydrophobic gas-permeable membrane, which affects the durability of the gas sensor.

### SUMMARY

A gas-permeable package lid of a chip package structure and a manufacturing method thereof are provided in the disclosure, in which the gas-permeable package lid of the chip package structure and the manufacturing method thereof may strengthen the bonding of a hydrophobic gas-permeable membrane and improve the reliability of element.

The gas-permeable package lid of the chip package structure of the disclosure includes a lid body, an air hole, and a hydrophobic gas-permeable membrane. The lid body is integrally formed with an encapsulation material, and has a body portion and multiple anchors. The air hole penetrates through the body portion of the lid body. The hydrophobic gas-permeable membrane is bonded to the lid body, and has a shielding part shielding the air hole and an embedded part embedded in the lid body. The embedded part has an upper surface and a lower surface. The upper surface and the lower surface respectively have multiple recesses. The anchors are respectively located in the recesses.

The manufacturing method of the gas-permeable package lid of the chip package structure of the disclosure includes the following operation. A hydrophobic gas-permeable membrane is provided. An encapsulation material in liquid form is cured into a lid body. The hydrophobic gas-permeable membrane is bonded to the lid body. The lid body has a body portion and multiple anchors. An air hole is formed in the lid body and penetrates through the body portion of the lid body. The hydrophobic gas-permeable membrane has a shielding part shielding the air hole, and an embedded part embedded in the lid body. The embedded part has an upper surface and a lower surface. The upper surface and the lower surface respectively have multiple recesses. The anchors are respectively located in the recesses.

### BRIEF DESCRIPTION OF THE DRAWINGS

FIG. 1A is a top schematic diagram of a gas-permeable package lid of a chip package structure applied to a chip package structure according to an embodiment of the disclosure.

FIG. 1B is a cross-sectional schematic diagram of FIG. 1A along line I-I.

FIG. 2A is a scanning electron microscope (SEM) image of the chip package structure adopting the architecture of FIG. 1A.

FIG. 2B is a scanning electron microscope image of the area A1 in FIG. 2A.

FIG. 2C is a scanning electron microscope image of the area A2 in FIG. 2B.

FIG. 2D is a scanning electron microscope image of the area A3 in FIG. 2A.

FIG. 2E is a scanning electron microscope image of the area A4 in FIG. 2D.

FIG. 3 is a top schematic diagram of the hydrophobic gas-permeable membrane of the gas-permeable package lid of the chip package structure in FIG. 1B at the material stage.

FIG. 4 is a top schematic diagram of another embodiment of the hydrophobic gas-permeable membrane of the gas-permeable package lid of the chip package structure in FIG. 1B at the material stage.

FIG. 5 is a cross-sectional schematic diagram of a gas-permeable package lid of a chip package structure applied to a chip package structure according to another embodiment of the disclosure.

FIG. 6A to FIG. 6E are cross-sectional schematic diagrams of the process of a manufacturing method of a gas-permeable package lid of a chip package structure according to an embodiment of the disclosure.

### DETAILED DESCRIPTION OF DISCLOSED EMBODIMENTS

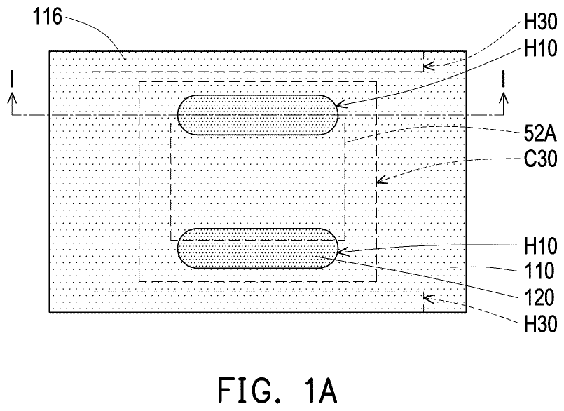

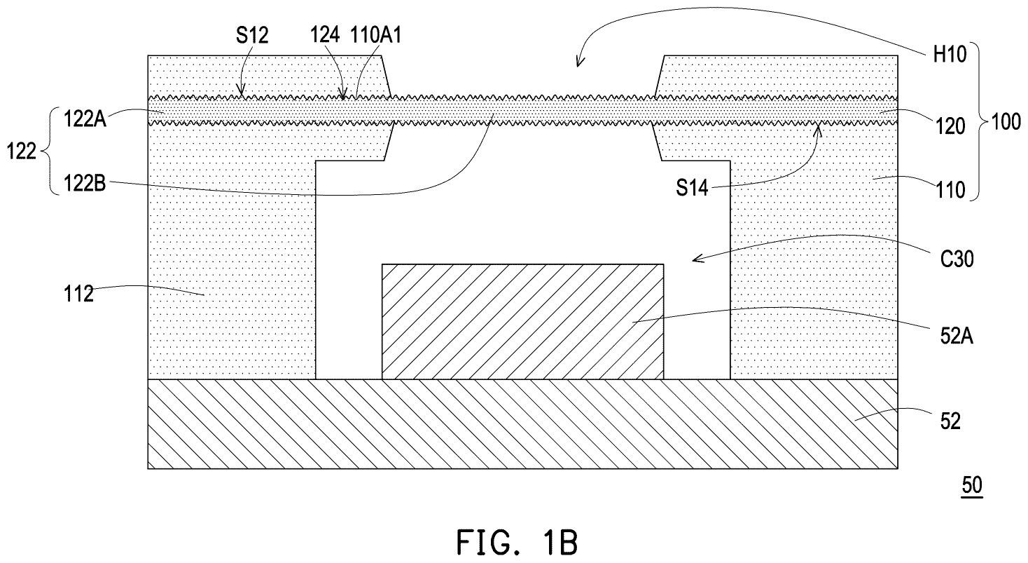

FIG. 1A is a top schematic diagram of a gas-permeable package lid of a chip package structure applied to a chip package structure according to an embodiment of the disclosure. FIG. 1B is a cross-sectional schematic diagram of FIG. 1A along line I-I. Referring to FIG. 1A and FIG. 1B, the chip package structure 50 using the gas-permeable package lid 100 of the chip package structure 50 of this embodiment includes a base 52 and a chip 52A. In this embodiment, the gas-permeable package lid 100 is assembled with the base 52 and covers the chip 52A. A cavity C30 is formed between the gas-permeable package lid 100 and the base 52. The gas-permeable package lid 100 includes a lid body 110, an air hole H10, and a hydrophobic gas-permeable membrane 120. The lid body 110 is integrally formed with an encapsulation material 110A, and has a body portion 112 and multiple anchors 110A1. The air hole H10 penetrates through the body portion 112 of the lid body 110.

The hydrophobic gas-permeable membrane 120 is bonded to the lid body 110, and has a shielding part 122B shielding the air hole H10 and an embedded part 122A embedded in the lid body 110. The chamber C30 communicates with the outside through the hydrophobic gas-permeable membrane 120 and the air hole H10. Through the function of the hydrophobic gas-permeable membrane 120, gas from the outside may enter the chamber C30 and contact the chip 52A, but moisture and other foreign matter cannot enter the chamber C30. The embedded part 122A of the hydrophobic gas-permeable membrane 120 has an upper surface S12 and a lower surface S14. The upper surface S12 and the lower surface S14 respectively have multiple recesses 124. The anchors 110A1 are respectively located in the recesses 124, that is, each of the recesses 124 has an anchor 110A1. In FIG. 1A, the dimensions of the anchor 110A1 and the recess 124 are enlarged for easy understanding, and do not represent actual dimensions. The recesses 124 located on the upper surface S12 may communicate with the recesses 124 located on the lower surface S14, and the channels partially communicated with the upper and lower surfaces S12 and S14 may be interleaved, so several of the recesses 124 of the upper surface S12 may communicate with several of the recesses 124 of the lower surface S14.

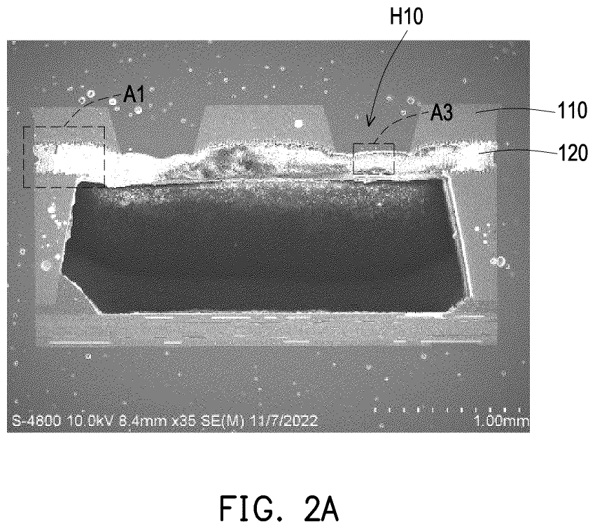

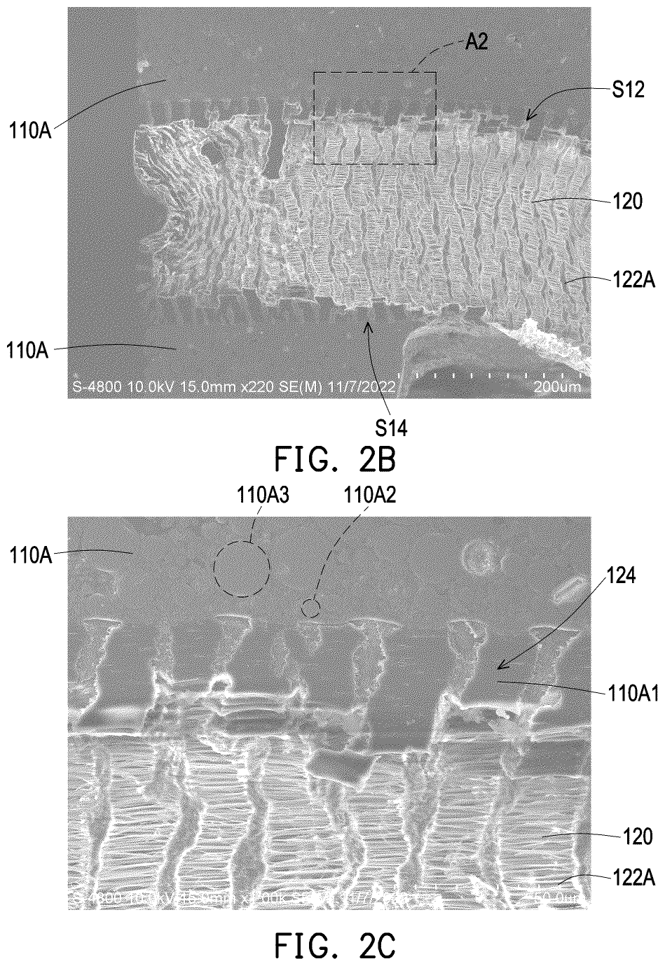

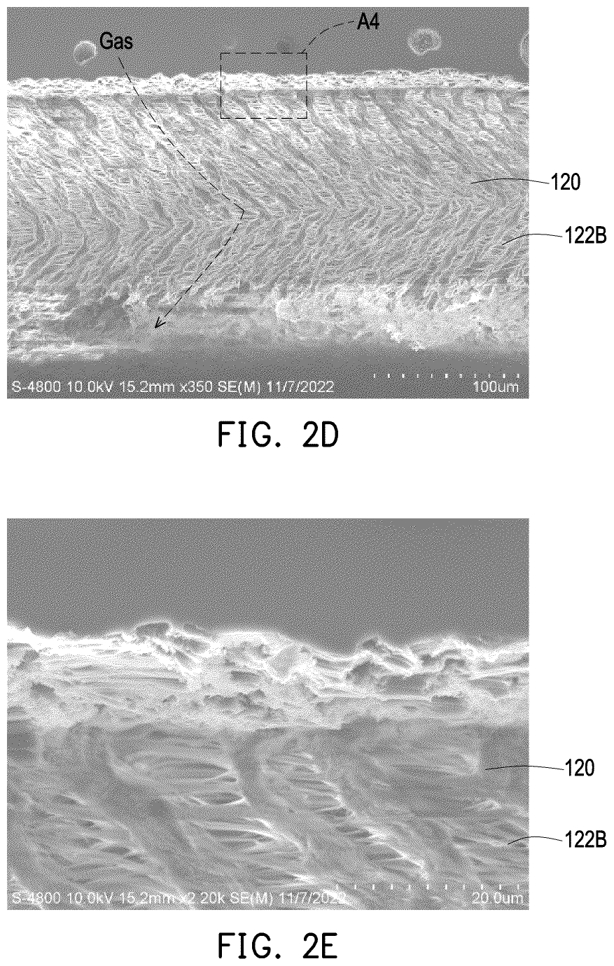

FIG. 2A is a scanning electron microscope image of the chip package structure adopting the architecture of FIG. 1A. As may be seen in FIG. 2A, two sides of the hydrophobic gas-permeable membrane 120 are embedded in the lid body 110, that is, corresponding to the embedded part 122A shown in FIG. 1B embedded in the lid body 110. A portion of the hydrophobic gas-permeable membrane 120 shields the air hole H10, that is, corresponding to the shielding part 122B shown in FIG. 1B shielding the air hole H10.

FIG. 2B is a scanning electron microscope image of the area A1 in FIG. 2A. FIG. 2C is a scanning electron microscope image of the area A2 in FIG. 2B. It may be seen from FIG. 2B and FIG. 2C that the upper surface S12 and the lower surface S14 of the embedded part 122A of the hydrophobic gas-permeable membrane 120 have multiple recesses 124. The encapsulation material 110A has multiple anchors 110A1 embedded in the recesses 124. This is because, when the lid body 110 (marked in FIG. 1B) is formed by a high-temperature liquefaction compression molding mechanism (e.g., injection molding) and embedded in the hydrophobic gas-permeable membrane 120, the encapsulation material 110A in liquid form permeates into the irregular pores of the hydrophobic gas-permeable membrane 120 and expands the pores into recesses 124. The encapsulation material 110A also forms the anchors 110A1 in the recesses 124 after curing. Through the bonding of the anchor 110A1 and the recess 124, the encapsulation material 110A may tightly grasp the hydrophobic gas-permeable membrane 120 to produce a good fixing effect. In other words, the recesses 124 of the hydrophobic gas-permeable membrane 120 are openings formed on the upper surface S12 and the lower surface S14 by the pores penetrating the hydrophobic gas-permeable membrane 120, so the recess 124 on the upper surface S12 communicates with the recess 124 on the lower surface S14; that is, the diameter of each of the recesses 124 is the same as the diameter of each of the irregular pores. For example, when the irregular pores are between 0.03 micron and 1 micron, the pore size of the recess 124 is also between 0.03 micron and 1 micron. In addition, the channels from the irregular pores of the hydrophobic gas-permeable membrane 120 penetrating through the upper surface S12 and the lower surface S14 may be interleaved. When the channels of the irregular pores are interleaved, several of the recesses 124 on the upper surface S12 and several of the recesses 124 on the lower surface S14 may communicate with each other.

The interface structure of the gas-permeable package lid 100 (marked in FIG. 1B) of an embodiment of the disclosure is shown in FIG. 2B, including the encapsulation material 110A and the hydrophobic gas-permeable membrane 120. The upper surface S12 and the lower surface S14 of the hydrophobic gas-permeable membrane 120 respectively have multiple recesses 124. The encapsulation material 110A has multiple anchors 110A1 embedded in the recesses 124. In this embodiment, there are encapsulation materials 110A on both upper and lower sides of the hydrophobic gas-permeable membrane 120, but in other embodiments of the disclosure, the encapsulation material 110A may only be located on one side of the hydrophobic gas-permeable membrane 120.

In this embodiment, the encapsulation material 110A has multiple first particles 110A2 and multiple second particles 110A3. The particle size of each of the first particles 110A2 is between 3 microns to 10 microns, and the particle size of each of the second particles 110A3 is between 20 microns to 35 microns. In another embodiment, the encapsulation material 110A may only have the first particles 110A2, and the particle size of each of the first particles 110A2 is between 3 microns to 10 microns. In other embodiments, the encapsulation material 110A may only have a single type of particles in another particle size, or may have more types of particles.

FIG. 2D is a scanning electron microscope image of the area A3 in FIG. 2A. FIG. 2E is a scanning electron microscope image of the area A4 in FIG. 2D. It may be seen from FIG. 2D and FIG. 2E that the hydrophobic gas-permeable membrane 120 has multiple irregular pores for gas to flow from one surface of the hydrophobic gas-permeable membrane 120 to the other surface. Since the irregular pores are not entirely located in a single cross-sectional position, only the gas flowing direction indicated by the dashed line is used to illustrate the irregular pores in FIG. 2D. The irregular pores may also be referred to as gas permeation channels communicated with the two surfaces of the hydrophobic gas-permeable membrane 120. In this embodiment, the diameter of each of the irregular pores is between 0.03 micron to 1 micron.

Referring to FIG. 1A and FIG. 1B, in the chip package structure 50 of this embodiment, because the lid body 110 is integrally formed, that is, when the lid body 110 is formed, the embedded part 122A of the hydrophobic gas-permeable membrane 120 is also embedded in the lid body 110, so after the lid body 110 is formed, the hydrophobic gas-permeable membrane 120 is also fixed. In this way, it may be ensured that the hydrophobic gas-permeable membrane 120 does not fall off, thereby improving the reliability of the gas-permeable package lid 100 of the chip packaging structure 50. In addition, no additional process is required to fix the hydrophobic gas-permeable membrane 120, which may reduce manufacturing process and material costs.

In this embodiment, the air hole H10 is located above the chip 52A, and the hydrophobic gas-permeable membrane 120 is suspended above the chip 52A. In this way, the chip 52A may quickly respond to external changes. In addition, the chip 52A of this embodiment may be a gas sensing chip.

In this embodiment, the hydrophobic gas-permeable membrane 122 has an embedded part 122A embedded in the lid body 110 and a shielding part 122B shielding the air hole H10. In this embodiment, the lid body 110 has a body portion 112 surrounding the air hole H10.

In addition, the hydrophobic gas-permeable membrane 120 of this embodiment may also have multiple flow holes H30, the flow holes H30 penetrate through the embedded part 122A of the hydrophobic gas-permeable membrane 122 and are adjacent to the edge of the hydrophobic gas-permeable membrane 122, and are also located at the edge of the hydrophobic gas-permeable membrane 120. A portion of the lid body 110 passes through the flow hole H30, and the flow hole H30 is embedded in the lid body 110. On the other hand, the lid body 110 also has multiple notch-anchored portions 116 located in the body portion 112. The notch-anchored portions 116 are respectively located in the flow holes H30 of the hydrophobic gas-permeable membrane 120. The notch-anchored portion 116 of the lid body 110 is locked in the flow hole H30 of the hydrophobic gas-permeable membrane 120, so it may also play the role of clamping and preventing the displacement of the hydrophobic gas-permeable membrane 120. The flow holes H30 of this embodiment are, for example, located on two opposite sides of the gas-permeable package lid 100.

In this embodiment, the surface of a portion of the hydrophobic gas-permeable membrane 120 around the air hole H10 facing and facing away from the chip 52A is covered by the lid body 110. That is, the portion of the hydrophobic gas-permeable membrane 120 around the air hole H10 is clamped from the top and bottom by the lid body 110. On the other hand, the embedded part 122A of the hydrophobic gas-permeable membrane 120 is located around the air hole H10, and the surface of the embedded part 112A facing and facing away from the chip 52A is covered by the lid body 110, while the surface of the shielding part 122B facing and facing away from the chip 52A of the hydrophobic gas-permeable membrane 120 is not covered by the lid body 110.

In this embodiment, the hydrophobic gas-permeable membrane 120 is coated with polytetrafluoroethylene, so it is hydrophobic and gas permeable. In this embodiment, the material of the gas-permeable package lid 100 is, for example, plastic.

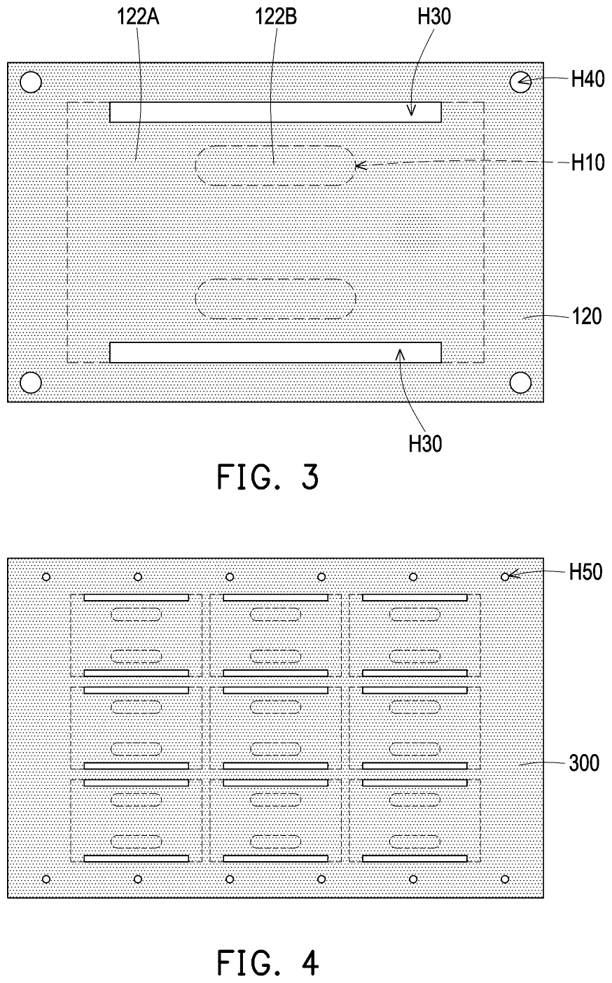

FIG. 3 is a top schematic diagram of the hydrophobic gas-permeable membrane of the gas-permeable package lid of the chip package structure in FIG. 1B at the material stage. Referring to FIG. 1A and FIG. 3, the hydrophobic gas-permeable membrane 120 of this embodiment has a larger size before being embedded in the lid body 110. In addition to the aforementioned flow holes H30, there are multiple process positioning holes H40 around the hydrophobic gas-permeable membrane 120 for positioning during the manufacturing process. After the hydrophobic gas-permeable membrane 120 is embedded in the lid body 110, the portion of the hydrophobic gas-permeable membrane 120 protruding from the lid body 110 is cut off, and the process positioning hole H40 is also cut off.

FIG. 4 is a top schematic diagram of another embodiment of the hydrophobic gas-permeable membrane of the gas-permeable package lid of the chip package structure in FIG. 1B at the material stage. Referring to FIG. 4, the hydrophobic gas-permeable membrane 300 of this embodiment is similar to the hydrophobic gas-permeable membrane 120 of FIG. 3, the difference is that a sheet of hydrophobic gas-permeable membrane 300 is distributed with nine hydrophobic gas-permeable membranes 120 as shown in FIG. 1B, which is suitable for batch production. The periphery of the hydrophobic gas-permeable membrane 300 also has multiple process positioning holes H50 for positioning during the manufacturing process.

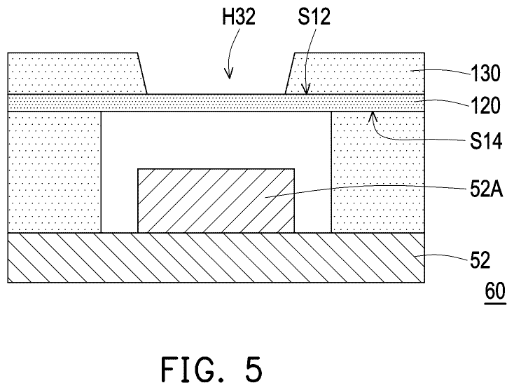

FIG. 5 is a cross-sectional schematic diagram of a gas-permeable package lid of a chip package structure applied to a chip package structure according to another embodiment of the disclosure. For clarity, the undulations on the surface of the hydrophobic gas-permeable membrane 120 in FIG. 5 are not enlarged. Referring to FIG. 5, the chip package structure 60 of this embodiment is basically the same as the chip package structure 50 of FIG. 1B, and only the differences between the two are described herein. The upper surface S12 of a portion of the hydrophobic gas-permeable membrane 120 of this embodiment around the air hole H32 facing away from the chip 52A is covered by the lid body 130, and the lower surface S14 of a portion of the hydrophobic gas-permeable membrane 120 around the air hole H32 facing the chip 52A is not covered by the lid body 130. Even so, other portions of the hydrophobic gas-permeable membrane 120 are still embedded in the lid body 110, so the hydrophobic gas-permeable membrane 120 may still be firmly positioned. In other words, the hydrophobic gas-permeable membrane 120 may be divided into two areas, such as the shielding part and the embedded part. The shielding part of the hydrophobic gas-permeable membrane 120 is the area that shields the air hole H32 and is not covered by the lid body 130, and the embedded part is the area embedded in the lid body 130 (covered by the lid body 130).

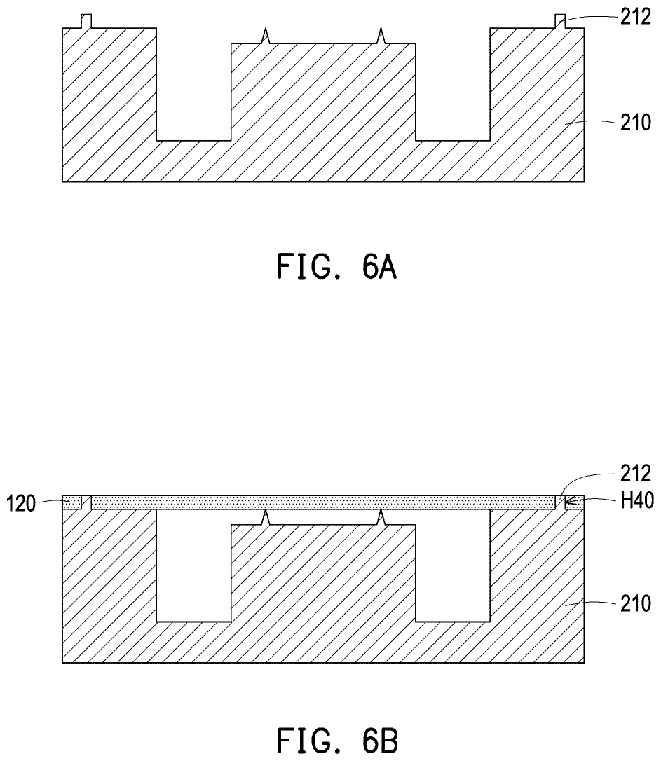

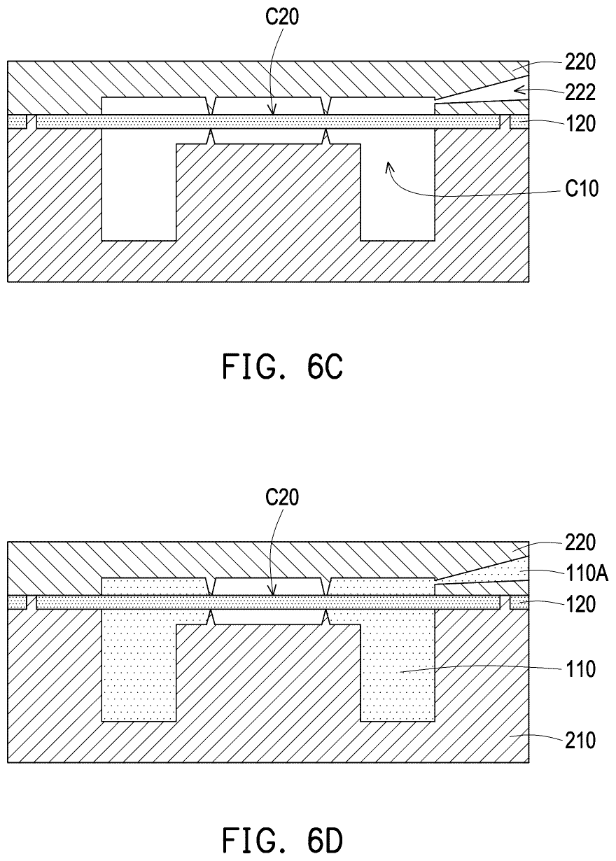

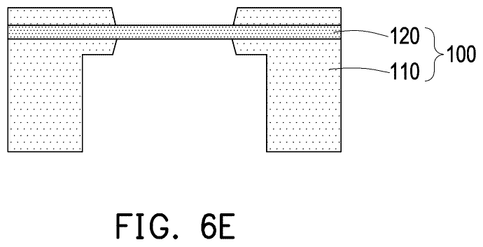

FIG. 6A to FIG. 6E are cross-sectional schematic diagrams of the process of a manufacturing method of a gas-permeable package lid of a chip package structure according to this embodiment. For clarity, the undulations on the surface of the hydrophobic gas-permeable membrane 120 in FIG. 6B to FIG. 6E are not enlarged. The manufacturing method of the gas-permeable package lid of the chip package structure in this embodiment includes at least the steps shown in FIG. 6B and FIG. 6D. Referring to FIG. 6B, a hydrophobic gas-permeable membrane 120 is provided. Next, referring to FIG. 6D, the encapsulation material 110A in liquid form is cured to form a lid body 110, in which the hydrophobic gas-permeable membrane 120 is bonded to the lid body 110. An air hole H10 is formed in the lid body 110 and penetrates through the lid body 110. The hydrophobic gas-permeable membrane 120 shields the air hole H10. As shown in FIG. 2B, the surface of the hydrophobic gas-permeable membrane 120 has multiple recesses 124, and the encapsulation material 110A has multiple anchors 110A1 embedded in the recesses 124.

In the manufacturing method of the gas-permeable package lid of the chip package structure in this embodiment, because the hydrophobic gas-permeable membrane 120 is also fixed when the lid body 110 is integrally formed, no additional process is required to fix the hydrophobic gas-permeable membrane 120, which may reduce manufacturing process and material costs.

In this embodiment, the step of curing the encapsulation material 110A in liquid form into the lid body 110 is to perform an insert molding process, but the disclosure is not limited thereto.

Hereinafter, other specific steps of the manufacturing method of the gas-permeable package lid of the chip package structure of this embodiment are illustrated with examples, but the disclosure is not limited thereto. Referring to FIG. 6A, firstly, a lower mold 210 is provided, in which the lower mold 210 has multiple positioning pins 212. Next, referring to FIG. 6B, the hydrophobic gas-permeable membrane 120 is positioned on the lower mold 210 by the positioning pins 212. Specifically, the positioning pin 212 passes through the process positioning hole H40 of the hydrophobic gas-permeable membrane 120 to position the hydrophobic gas-permeable membrane 120. Next, referring to FIG. 6C, an upper mold 220 and the lower mold 210 are clamped to form a molding cavity C10 communicating with an injection channel 222 in the upper mold 220 and the lower mold 210. The upper mold 220 and the lower mold 210 also form a closed cavity C20. The closed cavity C20 is isolated from the molding cavity C10 and the injection channel 222. In this embodiment, the injection channel 222 is located in the upper mold 220, but the disclosure is not limited thereto.

Next, referring to FIG. 6D, the encapsulation material 110A in liquid form is injected from the injection channel 222 into the molding cavity C10 and cured to form the lid body 110. Since the closed cavity C20 is isolated from the molding cavity C10 and the injection channel 222, the encapsulation material 110A does not enter the closed cavity C20, and the air hole H10 is formed in the closed cavity C20. Finally, referring to FIG. 6E, the lid body 110 is stripped.

For example, during the molding process, the upper mold 220 and the lower mold 210 are heated to between 155° C. and 185° C., the pressure range when injecting the encapsulation material 110A in liquid form is between 410 psi and 515 psi, and the pressure duration is between 16 seconds and 32 seconds.

In summary, in the manufacturing method of the chip package structure and its gas-permeable package lid of the disclosure, the hydrophobic gas-permeable membrane is embedded in the integrally formed lid body, and the positioning of the hydrophobic gas-permeable membrane is also completed when the lid body is formed. No additional positioning structure is required and the hydrophobic gas-permeable membrane does not easily fall off, thereby the manufacturing process is reduced and material costs are also relatively low.

What is claimed is:

1. A gas-permeable package lid of a chip package structure, applied to the chip package structure, the gas-permeable package lid of the chip package structure comprising:
a lid body, integrally formed with an encapsulation material, and having a body portion and a plurality of anchors;
an air hole, penetrating through the body portion of the lid body; and
a hydrophobic gas-permeable membrane, bonded to the lid body, and having a shielding part shielding the air hole and an embedded part embedded in the lid body, wherein the embedded part has an upper surface and a lower surface, the upper surface and the lower surface respectively have a plurality of recesses,
wherein, the anchors are respectively located in the recesses.

2. The gas-permeable package lid of the chip package structure according to claim 1, wherein the chip package structure further comprises a base and a chip disposed on the base, the air hole is located above the chip, and the hydrophobic gas-permeable membrane is suspended above the chip.

3. The gas-permeable package lid of the chip package structure according to claim 2, wherein the chip is a gas sensing chip.

4. The gas-permeable package lid of the chip package structure according to claim 1, wherein the hydrophobic gas-permeable membrane further comprises a plurality of flow holes, the flow holes penetrate through the embedded part of the hydrophobic gas-permeable membrane and are adjacent to an edge of the hydrophobic gas-permeable membrane, the lid body further comprises a plurality of notch-anchored portions, the notch-anchored portions are respectively located in the flow holes of the hydrophobic gas-permeable membrane.

5. The gas-permeable package lid of the chip package structure according to claim 1, wherein the chip package structure further comprises a base and a chip disposed on the base, the hydrophobic gas-permeable membrane is suspended above the chip, a surface of the shielding part facing the chip and a surface of the shielding part facing away from the chip of the hydrophobic gas-permeable membrane are not covered by the lid body.

6. The gas-permeable package lid of the chip package structure according to claim 1, wherein the hydrophobic gas-permeable membrane further comprises a plurality of flow holes penetrating through the hydrophobic gas-permeable membrane, the flow holes are adjacent to an edge of the hydrophobic gas-permeable membrane and are embedded in the lid body.

7. The gas-permeable package lid of the chip package structure according to claim 6, wherein the flow holes are located on two opposite sides of the air hole.

8. The gas-permeable package lid of the chip package structure according to claim 1, wherein the hydrophobic gas-permeable membrane is coated with polytetrafluoroethylene.

9. The gas-permeable package lid of the chip package structure according to claim 1, wherein the chip package structure further comprises a base and a chip disposed on the base, wherein the hydrophobic gas-permeable membrane is suspended above the chip, a surface of a portion of the hydrophobic gas-permeable membrane around the air hole facing the chip is not covered by the lid body, and a surface of a portion of the hydrophobic gas-permeable membrane around the air hole facing away from the chip is covered by the lid body.

10. The gas-permeable package lid of the chip package structure according to claim 1, wherein the hydrophobic gas-permeable membrane has a plurality of irregular pores for gas to flow from one surface of the hydrophobic gas-permeable membrane to another surface.

11. The gas-permeable package lid of the chip package structure according to claim 10, wherein a diameter of each of the irregular pores is between 0.03 micron to 1 micron.

12. The gas-permeable package lid of the chip package structure according to claim 1, wherein the encapsulation material has a plurality of particles, a particle size of each of the particles is between 3 microns to 10 microns.

13. The gas-permeable package lid of the chip package structure according to claim 1, wherein the encapsulation material has a plurality of first particles and a plurality of second particles, a particle size of each of the first particles is between 3 microns to 10 microns, a particle size of each of the second particles is between 20 microns to 35 microns.

14. The gas-permeable package lid of the chip package structure according to claim 1, wherein the recesses do not penetrate through the hydrophobic gas-permeable membrane.

15. A manufacturing method of a gas-permeable package lid of a chip package structure, comprising:
providing a hydrophobic gas-permeable membrane; and
curing an encapsulation material in liquid form into a lid body, wherein the hydrophobic gas-permeable membrane is bonded to the lid body, the lid body has a body portion and a plurality of anchors, an air hole is formed in the lid body and penetrates through the body portion of the lid body, the hydrophobic gas-permeable membrane has a shielding part shielding the air hole and an embedded part embedded in the lid body, the embedded part has an upper surface and a lower surface, the upper surface and the lower surface respectively have a plurality of recesses, and the anchors are respectively located in the recesses.

16. The manufacturing method of the gas-permeable package lid of the chip package structure according to claim 15, wherein curing the encapsulation material in liquid form into the lid body is performing an insert molding process.

17. The manufacturing method of the gas-permeable package lid of the chip package structure according to claim 15, wherein curing the encapsulation material in liquid form into the lid body comprises:
providing a lower mold, wherein the lower mold has a plurality of positioning pins;
positioning the hydrophobic gas-permeable membrane on the lower mold by the positioning pins;
clamping an upper mold and the lower mold to form a molding cavity communicating with an injection channel in the upper mold and the lower mold, wherein the upper mold and the lower mold further form a closed cavity, the closed cavity is isolated from the molding cavity and the injection channel;
injecting the encapsulation material in liquid form from the injection channel into the molding cavity and curing the encapsulation material in liquid form to form the lid body, wherein the air hole is formed in the closed cavity; and
stripping the lid body.

18. The manufacturing method of the gas-permeable package lid of the chip package structure according to claim 15, wherein a plurality of flow holes of the hydrophobic gas-permeable membrane are located at an edge of the hydrophobic gas-permeable membrane, a plurality of notch-anchored portions of the lid body are respectively located in the flow holes.

19. The manufacturing method of the gas-permeable package lid of the chip package structure according to claim 15, wherein the hydrophobic gas-permeable membrane is coated with polytetrafluoroethylene.

20. The manufacturing method of the gas-permeable package lid of the chip package structure according to claim 17, wherein the upper mold and the lower mold are heated to between 155° C. and 185° C., a pressure range when injecting the encapsulation material in liquid form is between 410 psi and 515 psi, and a pressure duration is between 16 seconds and 32 seconds.
